(19) United States
(12) Patent Application Publication
(10) Pub. No.: US 2005/0202038 A1
(43) Pub. Date: Sep. 15, 2005

(54) Composition and method for minimizing residual fecal matter in the perianal area

(76) Inventors: Roger E. Green, San Clamente, CA (US); Rollin M. Green, San Juan Capistrano, CA (US)

Correspondence Address: GABOR L. SZEKERES, 8141 E. KAI SER BLVD., SUITE 112, ANAHEIM HILLS, CA, 92808, US

(21) Appl. No.: 10/797,883

(22) Filed: Mar. 10, 2004

(51) Int. Cl. A61K 35/80; A61K 35/78

(52) U.S. Cl. 424/195.17; 424/738

(57) ABSTRACT

A combination of dried sea weed, preferably irish moss (chondrus crispus) and psyllium (plantago ovata, plantago psyllium) is administered to a person or domestic animal in a daily dose which in its broadest range comprises 58 miligram (mg) to 4.6 gram (g) of sea weed and 250 mg to 24 g of psyllium for the purpose of allowing the stool to make a clean exit from the body thereby eliminating or reducing residual fecal matter that adheres to the perianal area after the bowel movements of the person or domestic animal.

### BACKGROUND OF THE INVENTION

1. Field of the Invention

The present invention is in the field of compositions to be ingested internally by humans and domestic animals. More specifically the present invention pertains to a composition and method of administering the composition to humans or domestic animals for the purpose of minimizing, after each bowel movement, residual fecal matter in the perianal area of the person or domestic animal.

2. Brief Description of the Prior Art

In modem society myriads of products are available for daily use to increase personal hygiene, to render ordinary living more antiseptic and to eliminate undesirable body odors. Good personal hygiene also requires avoidance of soiling a person's underwear by cleansing the perianal area after each bowel movement. Nevertheless, unlike the myriads of products available for other personal hygiene purposes, the only product available and in regular use for cleansing the perianal area is toilet paper.

However, the practice of wiping the perianal area with toilet paper normally fails to completely cleanse the perianal area of residual fecal matter, some of which remains on the surface, in the crevices of the anus and attached to perianal hair. For some people this residual fecal matter can cause irritation with itching and burning sensation and can give rise to skin infections or other skin disorders. Moreover, wiping the perianal area with toilet paper also tends to transfer bacteria and some residual fecal matter to the person's hand. In the absence of adequate cleansing of the hands this residual trace of fecal matter can result in the transmission of serious infectious diseases, such as hepatitis A. Obese persons and paralyzed persons have particular difficulty in adequately cleansing the perianal area by the standard practice of wiping, and some persons pay inadequate attention to washing or otherwise cleansing their hands after wiping the perianal area with toilet paper.

In light of the foregoing, a significant advance in the field of personal hygiene, medicine and public health could be accomplished by reducing the residual fecal matter that remains in the perianal area after each bowel movement, and by eliminating or reducing the need for wiping with toilet paper or like material. As far as the present inventors are aware the prior art has not addressed the above-noted issues and has not provided any solutions or improvements over the ordinary practice of using toilet paper. For this reason the present inventors are not aware of any prior art that directly pertains to the present invention.

Compositions that contain one or possibly more of the ingredients included in the composition of the present invention are described or disclosed in the following U.S. Pat. No. 6,479,545 (Formulation for menopausal women); U.S. Pat. No. 6,455,557 (Method of reducing somnolence in patients treated with tizanidine); U.S. Pat. No. 6,361,651 (Chemically modified pulp fiber); U.S. Pat. No. 6,352,713 (Nutritional composition); U.S. Pat. No. 6,004,610 (Compositions of dietary fiber rich and low viscosity beverages); U.S. Pat. No. 4,911,937 (Chewable, peelable, layered soft nougat candies); U.S. Pat. No. 5,397,573 (Laxative compositions); U.S. Pat. No. 4,877,627 (Balanced fiber composition); U.S. Pat. No. 6,472,408 (Dimeric compounds); and U.S. Pat. No. 6,613,733 (Treating compositions comprising polysaccharides).

### SUMMARY OF THE INVENTION

In accordance with the present invention, a dried sea weed is selected from the group consisting of Red seaweed (Rhodophycae) of the Solieriaceae, Gigartinaceae, Furcellariaceae, Phyllophoraceae, Hypneaceae, Rhabdoniaceae, Rhodophyllidaceae families and irish moss (chondrus crispus). The selected seeweed or combination of seaweeds is administered together with psyllium (plantago ovata, plantago psyllium to a person or domestic animal in a daily dose which in its broadest range comprises 58 miligram (mg) to 4.6 gram (g) of sea weed, preferably irish moss, and 250 mg to 24 g of psyllium for the purpose of allowing the stool to make a clean exit from the body thereby eliminating or reducing residual fecal matter that adheres to the perianal area after each bowel movement of the person or domestic animal. For small domestic animals, such as dogs kept as household pets, the daily dose of sea weed (irish moss) and psyllium tends to be in the lower part of the above-noted broad range.

The combination of the above-noted components can be provided and administered to humans or domestic animals as a single composition wherein the components are preferably admixed, or the two components can be provided and administered separately. Each of the components, or the composition containing the two components may optionally include additional substances, such as pharmaceutically acceptable excipients, vitamins, minerals, pharmaceuticals, flavoring agents or nutrients, the foregoing list being exemplary rather than limiting in nature.

Data obtained from human subjects in a 6 weeks long study demonstrated that residual fecal matter in the perianal area is eliminated or significantly reduced when the subjects were administered the combination of irish moss and psyllium in accordance with the present invention.

### DETAILED DESCRIPTION OF THE INVENTION

A combination of dried sea weed and psyllium is administered to a human subject or to a domestic animal in accordance with the present invention in order to eliminate or reduce the residual fecal matter that stays in the perianal area after bowel movement.

The sea weeds suitable for this purpose are of the Red seaweed (Rhodophycae) of the Solieriaceae, Gigartinaceae, Furcellariaceae, Phyllophoraceae, Hypneaceae, Rhabdoniaceae and Rhodophyllidaceae families. Examples are the following species, Euchema cottonii and E.spinosum, Gigartina and Furcellaria and irish moss (chondrus crispus), or their powdered constituents. Presently the use of irish moss in the invention is preferred. Dried sea weed is available commercially and in bulk form from a number of suppliers. The presently preferred sea weed component of the invention, irish moss, is available in a dried powdered form, for example, from drug stores and herbalists and is produced in quanity by the harvesting of seaweed from around the world in places such as Ireland, the American Northeastern coast, Chile and the Orient. In the ensuing description reference is primarily made to irish moss, although it should be understood that the other dried sea weeds listed above are also suitable for use in the present invention, and that the several sea weeds can be used in combination with one another or in combination with the presently preferred irish moss and in substantially in the same dosage ranges of total sea weed content as the preferred example of irish moss.

The daily dose of irish moss to a human subject is in the range of 58 mg to 4.6 g. A more preferred range is 145 mg to 2.32 g per day, and a still more preferred range is 290 mg to 1.16 g per day. For children and for small domestic animals, such as dogs, the daily dose would be adjusted to be in the lower part of the above-noted ranges. The dried sea weed, preferably irish moss, is preferably administered in capsules to avoid the subject experiencing an unpleasant taste or dried powder in the mouth.

Encapsulation of dietary supplements, pharmaceuticals, vitamins, minerals and the like is a well developed technology and need not be described here. The dried sea weed, preferably irish moss, in whatever form it is administered, may be admixed with other optional ingredients, such as vitamins, minerals, flavoring agents, pharmaceuticals or nutrients.

Psyllium as used in the pharmaceutical and/or nutritional industry is the husk of a plant (plantago ovata, plantago psyllium) that grows in India in the Middle-East and elsewhere. Psyllium is readily available commercially, for example it can be purchased in drug stores in the United States. Because it is a husk, it is best consumed by first admixing it with water in order to avoid inhalation of light dust. Alternatively, the psyllium husk can also be broken into smaller particles and encapsulated in a manner well known in the art. Psyllium, in whatever form, admixed with water or in capsules, is also administered in accordance with the present invention to human subjects or to domestic animals in combination with the above-described sea weed. Psyllium is administered in a daily dose range of 250 mg to 24 g. This amount corresponds to approximately ¼ of a teaspoon to 5 tablespoons per day. A more preferred daily dose range of psyllium is 250 mg to 16 g, and a still more preferred daily dose range is 500 mg to 12 g. Just like the sea weed described above, the psyllium can also be admixed with optional ingredients, such as vitamins, minerals, flavoring agents, pharmaceuticals or nutrients. As in the case of sea weed, for children or small domestic animals the daily dose of psyllium is also adjusted in the lower part of the above-noted ranges.

Generally speaking, the actual daily dose of the combination of the sea weed and psyllium components in accordance with the present invention that is effective for any given individual is best determined by simple routine experimentation with regard to that individual. Such experimentation is routine in the nutritional component and pharmaceutical industry when the optimal effective dose of an agent or drug for any given individual is sought.

Instead of providing separately for ingestion the two essential components, sea weed and psyllium, of the present invention, the two could also be combined and admixed in particularized forms, optimally to be ingested when mixed with water, or admixed and placed in appropriate soft or hard capsules. The above noted optional and additional components may also be contained in these admixed formulations.

The combination of the foregoing two components, whether provided and administered separately or in an admixture, with or without further optional ingredients, is best considered a dietary supplement. For the purposes of administering the dietary supplement of the present invention to a domestic animal, such as a dog, it may become particularly desirable to combine one or both components of the combination with a flavor enhancer or flavorful nutrient, so as to render the combination more palatable for the animal.

A study that proved the effectiveness of the dietary supplement of the present invention involved 12 healthy persons (7 male and 5 female in the age range of 12 to 87 years) of whom 11 completed the study. The study involved a 15 days long control period wherein the subjects were on a normal diet and were not administered the dietary supplement comprising the combination of sea weed and psyllium of the present invention. During this control period the subjects were asked to record the number of wipes (with ordinary commercial toilet paper) that they performed after each bowel movement to obtain a final clean wipe. The average number of wipes during this control period was 5.6 per bowel movement. In a subsequent 4 weeks long period the same subjects remained on a normal diet and also ingested each morning with breakfast one capsule containing 580 mg of dried irish moss and one tablespoon (approximately 6 g) of psyllium mixed with water. During this same period, just as during the control period, perianal fecal contamination was effectively measured by recording the number of wipes with toilet paper necessary to produce the first clean wipe. The average number of wipes during this test period in accordance with the present invention was 1.6. There were also 187 “one wipe” episodes recorded during the study, that is bowel movements where the first wipe was already clean. No such “one wipe” episodes were recorded during the control study.

The foregoing represents a 71% decrease in the number of wipes per bowel movement necessary to produce the first clean wipe. This result is statistically significant and demonstrates much cleaner exit of stool than without the invention, and frequent clean exit that is virtually or completely free of leaving residual fecal matter in the perianal area.

The benefits to individuals and to the public from the present invention are significant. First, the general public and individuals will benefit from much improved perianal hygiene as a result of significantly reduced incidence of certain infectious diseases, such as hepatitis A, which are spread by fecal matter that is inadvertently transferred to and retained on persons' hands. A reduction in the incidents of hepatitis A can, in and of itself, be considered a very significant public health benefit.

Individuals will benefit from reduced incidents of skin problems, such as irritation itching and burning sensation that are caused by residual fecal matter in the perianal area. Obese persons and persons who are paralyzed or for some other health reason cannot reach the perianal area will benefit greatly from the invention. Infants and adults wearing diapers will also find the present invention very beneficial. Military personnel, while on maneuvers, and persons engaged in prolonged outdoor activity will find the use of the dietary supplement of the present invention advantageous because bowel movements will represent less of a burden.

Domestic animals, such as cats and dogs kept indoors as household pets frequently leave fecal matter on rugs by “skidding” on the rugs in an effort to alleviate perianal irritation and itching caused by residual fecal matter. These animals would also benefit from the dietary supplement of the present invention, and so would the homes housing such animals.

The present invention will also have an environmental impact, as the use of less toilet paper will require less cutting of trees to make paper and will also result in lesser bulk of sewage.

1. A dietary supplement for humans or domestic animals, comprising in combination:
a dried seaweed selected from the group consisting of Red seaweed (Rhodophycae) of the Solieriaceae, Gigartinaceae, Furcellariaceae, Phyllophoraceae, Hypneaceae, Rhabdoniaceae and Rhodophyllidaceae families and irish moss (chondrus crispus) in a measured amount comprising a daily dose, and
psyllium (plantago ovata, plantago psyllium) in a measured amount comprising a daily dose.

2. A dietary supplement in accordance with claim 1 where the measured amount comprising a daily dose of sea weed is 58 mg to 4.6 g, and where the measured amount comprising a daily dose of psyllium is 250 mg to 24 g.

3. A dietary supplement in accordance with claim 2 where the sea weed is irish moss.

4. A dietary supplement in accordance with claim 2 where the measured amount comprising a daily dose of sea weed is 145 mg to 2.32 g, and where the measured amount comprising a daily dose of psyllium is 250 mg to 16 g.

5. A dietary supplement in accordance with claim 4 where the sea weed is irish moss.

6. A dietary supplement in accordance with claim 2 where the measured amount comprising a daily dose of sea weed is 290 mg to 1.16 g, and where the measured amount comprising a daily dose of psyllium is 500 mg to 12 g.

7. A dietary supplement in accordance with claim 6 where the sea weed is irish moss.

8. A dietary supplement in accordance with claim 2 further comprising an ingredient selected from the group consisting of vitamins, minerals, flavoring agents, pharmaceuticals and nutrients.

9. A dietary supplement in accordance with claim 2 wherein the measured daily doses of sea weed and psyllium are not admixed with one another.

10. A dietary supplement in accordance with claim 2 wherein the daily doses of sea weed and psyllium are admixed with one another to form a single composition.

11. A dietary supplement comprising in combination:
dried irish moss (chondrus crispus) in a measured amount of 58 mg to 4.6 g forming a daily dose for a human being, and psyllium (plantago ovata, plantago psyllium) in a measured amount of 250 mg to 24 g forming a daily (lose for the human being.

12. A dietary supplement in accordance with claim 11 wherein the daily dose of dried irish moss is 145 mg to 2.32 g, and wherein the daily dose of psyllium is 250 mg to 16 g.

13. A dietary supplement in accordance with claim 11 wherein the daily dose of dried irish moss is 290 mg to 1.16 g, and wherein the daily dose of psyllium is 500 mg to 12 g.

14. A dietary supplement in accordance with claim 11 wherein the measured daily doses of sea weed and psyllium are not admixed with one another.

15. A dietary supplement in accordance with claim 11 wherein the measured daily doses of sea weed and psyllium are admixed with one another.

16. A method of administering to a human being or to a domestic. animal a dietary supplement comprising a combination of (1) a sea weed selected from the group consisting of Red seaweed (Rhodophycae) of the Solieriaceae, Gigartinaceae, Furcellariaceae, Phyllophoraceae, Hypneaceae, Rhabdoniaceae and Rhodophyllidaceae families and irish moss (chondrus crispus), and
(2) psyllium (plantago ovata, plantago psyllium) in a daily dose of 58 mg to 4.6 g of the sea weed and 250 mg to 24 g of psyllium, said combination being administered for the purpose of minimizing, after bowel movement, the amount of residual fecal matter in the perianal area of the human or of the domestic animal.

17. A method in accordance with claim 16 wherein the daily dose of the sea weed is 145 mg to 2.32 g, and the daily dose of psyllium is 250 mg to 24 g.

18. A method in accordance with claim 17 where the sea weed is irish moss.

19. A method in accordance with claim 16 wherein the daily dose of the sea weed is 290 mg to 1.16 g, and the daily dose of psyllium is 500 mg to 12 g.

20. A method in accordance with claim 19 where the sea weed is irish moss.

21. A method in accordance with claim 16 wherein the dietary supplement is administered to a human being.

22. A method in accordance with claim 16 wherein the dietary supplement is administered to a domestic animal.

23. A method in accordance with claim 16 wherein the sea weed and psyllium are not admixed prior to administration to the human or domestic animal.

24. A method in accordance with claim 16 wherein the sea weed and psyllium are admixed prior to administration to the human or domestic animal.
